(19) United States
(12) Patent Application Publication
(10) Pub. No.: US 2001/0055600 A1
(43) Pub. Date: Dec. 27, 2001

(54) LONG-WEARING COSMETIC COMPOSITIONS

(76) Inventor: AMIT R. SHAH, COMMACK, NY (US)

Correspondence Address: KENYON & KENYON, ONE BROADWAY, NEW YORK, NY, 10004, US

(21) Appl. No.: 09/248,524

(22) Filed: Feb. 9, 1999

(51) Int. Cl. A61K 6/00; A61K 7/00; A61K 7/021; A61K 7/025; A61K 7/06

(52) U.S. Cl. 424/401; 424/63; 424/64; 424/70.7

(57) ABSTRACT

The invention relates to long-wearing cosmetic compositions comprising an acrylic acid derived polymer or copolymer and at least one water soluble organic pigment. The polymer or copolymer can be in the form of an emulsion. The compositions are long lasting, water resistant and exhibit substantially indelible qualities. Further, the compositions will not smear, run or settle in the lines and creases of the skin. These compositions are useful as eyeliners, other cosmetic products, or as body paints.

### FIELD OF THE INVENTION

The invention relates to long-wearing cosmetic compositions. More specifically, the invention relates to long-wearing cosmetic compositions useful as eyeliners, cosmetic products or skin products that do not smear, run or settle in the lines and creases of the skin.

### BACKGROUND OF THE INVENTION

There is a strong demand among consumers for cosmetic products that last all day without the need for refreshing or touching up. The preferred cosmetic product is also easy to apply, and leaves a clear vivid color which lasts at least through the work day, and even into the evening. Given the hectic lifestyles of most consumers, however, providing such a product is not a simple task. Daily activity, particularly in the form of physical exercise, which is now so common, is not conducive to makeup retention. The combination of perspiration and body oils causes typical color products to routinely fade away with very little effort. In addition, the preferred product does not readily transfer from the place of application. Consumers are less tolerant of a lipstick if it leaves its color on a coffee cup, or a foundation which smudges onto the collar of a white blouse.

To obtain vivid colors in cosmetic compositions, various pigments and dyes are used. The cosmetic industry primarily uses inorganic pigments because they can withstand assaults on stability caused by water, oxidation, light and temperature extremes. Even though there are issues regarding their stability; they are, nonetheless, much more stable than natural pigments, in general. Notwithstanding the superior stability and general flexibility of inorganic pigments, such as metal oxides, consumers are now demanding the use of what are perceived to be “natural” products. For example, organic pigments are generally considered by the consumer to be natural and better than the use of inorganic pigments. However most, if not all, organic pigments have a tendency to bleed, fade, or otherwise deteriorate in a very short period of time. And, being water soluble, they have a tendency to run once they are applied to the skin. Therefore, the requirements of modern cosmetics are often not met by their use and, there is a continuing need to find ways of enhancing the stability of organic pigments.

There have been many reports in the industry as to methods for stabilizing organic pigments. However, to date, none have found widespread commercial application in the cosmetics industry, perhaps because many are designed for use only with a specific kind of pigment, and the resulting pigment is useful only with one particular kind of solvent system.

Cosmetic products, including those that are waterproof, have been developed which utilize water based acrylic polymers, such as those described in U.S. Pat. Nos. 3,697,643, 4,423,031, 5,165,915, and 5,356,627 incorporated herein by reference. These products contain inorganic pigments and often have other undesirable properties, such as smudging and flaking, associated with the use of these polymers. These shortcomings exist because the acrylic polymer is often incompatible with other components of the formulation. Further, eyeliners containing acrylic polymer run and cause fine lines and wrinkles in the skin to be accentuated. Thus, there continues to be a need for a cosmetic product that is not subject to running, flaking, or smudging, and retains its strong, non-fading color throughout the day.

Eyeliners as well as other cosmetic or skin products function to make up or enhance the beauty of the skin. In addition, cosmetic products can be used to draw on or decorate the skin to create what is commonly referred to as body art. Precise application of the cosmetic in these circumstances is critical to creating artistic work on the skin. Therefore, unlike present products such as eyeliners, a product is desired which does not clog in the applicator and prevent smooth clear lines. When cosmetic or skin products are used in this fashion, the consumer does not want the product to run or settle into the lines and creases of the skin. Further, the consumer wants a cosmetic or skin product that can be easily removed when desired. The present invention now provides such a product.

### SUMMARY OF THE INVENTION

The present invention provides long-wearing cosmetic compositions which are useful as eyeliners or, other eye or skin products. The compositions comprise an acrylic or methacrylic acid derived polymeric or copolymeric component in combination with at least one water soluble organic pigment. The long-wearing cosmetic composition is water resistant and stays on as applied (i.e., it is long lasting). It does not smudge, run or settle into the lines and creases of the skin. In addition, it is removable after use by using soap and water or any makeup remover that is known in the art. An example of such a makeup remover includes but is not limited to those that include cyclomethicone. Preferably, solvent based makeup removers are used to remove the compositions of the present invention.

A method for preparing a long-wearing cosmetic composition that is useful as an eyeliner and comprises combining the acrylic or methacrylic acid derived polymer or copolymer and the water soluble organic pigment is included in the present invention. An effective amount of the composition can be applied to the rim of the upper and lower eyelids to outline the eye. The eyeliner beautifies the eye by accentuating the outline of the eye. The present invention can also be used for creating body art on the skin by drawing on the skin with the long-wearing cosmetic compositions.

### DETAILED DESCRIPTION OF THE INVENTION

The present invention relates to long-wearing cosmetic compositions comprising an acrylic or methacrylic acid derived polymer or copolymer and one or more water soluble organic pigments. It was surprisingly found that the combination of acrylic or methacrylic acid derived polymers or copolymers with the water soluble organic pigment provided compositions that are water resistant, long lasting and exhibit substantially indelible qualities. In addition, the compositions unexpectedly do not run or bleed. The compositions are particularly useful as an eyeliner. However, the compositions can also be used in other cosmetic products and as body paint for body art. The compositions do not run because they form a film on the skin. The film formation is water resistant and prevents the eyeliner from settling into the fine lines of the surface of the skin. Additionally, as the compositions comprise a water soluble polymeric or copolymeric component, water soluble dyes are easily added to the composition.

A preferred acrylic acid derived polymer or copolymer is a high molecular weight (>100,000) and highly branched chain molecule. The polymeric or copolymeric component includes homopolymers and copolymers of acrylic acid, methacrylic acid, and esters of acrylic or methacrylic acid and salts thereof. The polymeric component is composed of monomeric elements having 1 to 18 carbon atoms. The monomeric elements of the polymer can include, for example, methylmethacrylate, butylacrylate, and combinations thereof.

In a preferred embodiment, the polymeric component is available commercially from Interpolymer Corporation, Canton, Mass., as a product named Syntran EX35-1™ which is an aqueous polymeric emulsion of ammonium acrylate or as Syntran EX33-9™ which is an acrylate copolymer. Both products comprise monomeric elements of methylacrylic acid (methacrylate), methylmethacrylate and butylacrylate. These types of polymers are also available from other sources.

In the preferred embodiment, the polymeric or copolymeric component is present in an amount of from about 5 to about 95 percent of the total weight of the composition, preferably about 10 to about 80 percent by weight of the composition, more preferably about 20 to about 50 percent. The organic pigment is in an amount of from about 1 to about 20 percent, preferably about 5 to about 10 percent by weight of the total composition.

The organic pigment component of the present invention is water soluble and comprises natural pigments, monomeric synthetic pigments, polymeric synthetic pigments or combinations thereof. Exemplary water soluble organic pigments, when the composition is not used in the eye area, are phthalocyanine blue and green pigments and azo-type red pigments such as naphthol red pigment. If they are water soluble, such as calcium hydrate. Other suitable pigments include D&C, and FD&C pigments. The water soluble organic pigment component also includes blends of other suitable organic pigments. Preferably, when the composition is used as a liner for the eye, the water soluble organic pigment is FD&C blue No.1, FD&C green No. 5, FD&C red No. 40, and FD&C yellow No. 5.

The long-wearing cosmetic compositions of the present invention are based on the use of water soluble organic pigments in combination with an acrylic acid derived polymer or copolymer. However, it may be desirable to include small amounts of additional pigments that are cosmetically acceptable, such as inorganic pigments or combinations of organic and inorganic pigments. Accordingly, the composition contains no more than about 1 to about 10 percent by weight of an inorganic pigment. Examples of useful inorganic additional pigments include iron oxides (yellow, red, brown or black), ferric ammonium ferrocyanide (blue), manganese violet, ultramarine blue, chrome oxide (green), talc, lecithin modified talc, zeolite, kaolin, lecithin modified kaolin, titanium dioxide (white) and mixtures thereof. Other useful additional pigments are pearlants such as mica, bismuth oxychloride and treated micas, such as titanated micas and lecithin modified micas. The use of other cosmetically acceptable additional pigments is limited to the extent that it does not interfere with the desired result achieved by the present invention.

Examples of useful additional pigments for the sensitive eye area are metallic oxides, such as titanium or iron oxides, bismuth oxychloride, carmine, chromium oxide or chromium hydroxide greens, ultramarines, ferric ferrocyanide, ferric ammonium ferrocyanide, or mica. It will be recognized that when the product is, for example, an eyeliner or other eye product, the pigment should be one which is approved for use in the eye area. Additional inorganic pigment concentrations will vary depending upon the color of the final product, but generally will be in the range of from about 0.1 to about 3 percent more preferably from about 1 to about 2 percent, by weight of the total composition, if used.

The compositions of the invention also includes any base that is aqueous or that is miscible in water. Thus, the medium is predominantly aqueous but it can also include solvents such as, for example, hydroalcohol, glycerin, and combinations thereof. Examples of acceptable hydroalcohols include, but are not limited to ethanol, propanol, or glycols such as butylene glycol or propylene glycol. While a large variety of polyols are capable of being used as the base, it is preferred that the polyol be a C2-C6 alcohol. Preferably, the base is butylene glycol.

The compositions of the invention may include one or more preservatives such as, for example, propyl paraben, butyl paraben, mixtures thereof, or isoforms thereof, as well as butyl hydroxy toluene or butyl hydroxy anisol (BHT or BHA).

The present invention also includes a flow-through nib-type pen for applying the composition. An added benefit of the compositions of the present invention is that they will not clog the wick of the pen as compositions containing titanium dioxide or other particulate pigments do. The long-wearing cosmetic composition of the present invention can also be applied using standard eyeliner applicators.

The present invention also includes a method of preparing long-wearing cosmetic compositions which comprises combining the acrylic acid derived polymer or copolymer and the water soluble organic pigment. The compositions of the present invention are useful as liquid eyeliners, other eye products, or as body paints. The compositions can be used for outlining the eye by applying the long-wearing cosmetic compositions around the rim of the eye. When used for the purpose of body art or tattoos, the compositions can be used to create long-lasting, yet temporary, tattoo-like designs on the skin. The compositions are drawn onto the skin using an applicator such as a brush, a wick type nib pen, or other similar type of device which will allow artistic designs to be drawn on the skin, possibly in conjunction with a stencil. The compositions of the present invention can also be in the form of eye or skin products whereby the product is used to create body art on the face around or in close proximity to the eye. For example, artistic designs around or near the eye can be drawn on the face as part of making up the face or decorating the face. Although it will be temporarily drawn on the skin, the art form can be retained up to a full day without smudging or running and can be removed with soap and water or any solvent based makeup remover.

The invention is further illustrated by the following non-limiting examples:

### EXAMPLES

An acrylic polymer component of the present invention is provided by Interpolymer Corporation, Canton, Mass., having the composition provided below. A formulation of the long-wearing cosmetic composition according to the present invention is provided below:

| | |
|---|---|
| Ingredient | Percent |

ACRYLIC COPOLYMER

| | |
|---|---|
| Water | 55.00 |
| Ammonium acrylate | 39.00 |
| copolymer | |
| Sodium lauryl ether sulfate | 1.25 |
| Butylene glycol | 4.25 |
| Methylparaben | 0.25 |
| Propylparaben | 0.25 |

LONG-WEARING COSMETIC COMPOSITION

| | |
|---|---|
| Acrylic copolymer | 90.00 |
| D & C Green No. 5 | 2.00 |
| FD & C Blue No. 1 | 1.75 |
| FD & C Red No. 40 | 1.50 |
| FD & C Yellow No. 5 | 2.00 |
| 1,3 Butylene glycol | 2.00 |
| Preservative | 0.75 |

The components are combined and mixed to homogeneity. The product so prepared is stable, and highly resistant to smearing, running or settling into the lines or creases of the skin.

What we claim is:

1. A long-wearing cosmetic composition comprising a polymeric component selected from the group consisting of an acrylic acid derived polymer or copolymer, acrylic acid ester derived polymer or copolymer, a methacrylic acid derived polymer or copolymer, and a methacrylic acid ester derived polymer or copolymer, and at least one water soluble organic pigment.

2. The composition of claim 1 wherein said polymeric component is present in an amount of from about 5 to about 95 percent by weight of composition.

3. The composition of claim 2 wherein said polymeric component comprises monomeric elements having about 1 to 18 carbon atoms.

4. The composition of claim 3 wherein at least one of said monomeric elements are selected from the group consisting of methacrylate, methylmethacrylate, butylacrylate, and combinations thereof.

5. The composition of claim 4 wherein said polymeric component is ammonium acrylate.

6. The composition of claim 1 wherein said water soluble organic pigment is present in an amount of from about 1 to about 20 percent by weight of composition.

7. The composition of claim 1 wherein said water soluble organic pigment is selected from the group consisting of a natural pigment, a monomeric synthetic pigment, a polymeric synthetic pigment, and combinations thereof.

8. The composition of claim 7 wherein said water soluble organic pigment is selected from the group consisting of FD&C blue No.1, FD&C green No. 5, FD&C red No. 40, FD&C yellow No. 5, and combinations thereof.

9. The composition of claim 1 further comprising an inorganic pigment present in an amount of no more than about 1 to about 10 percent by weight of the composition.

10. A long-wearing cosmetic eyeliner or body paint composition comprising: a) from about 5 to about 95 weight % of a polymeric component selected from the group consisting of an acrylic acid derived polymer or copolymer, an acrylic acid ester derived polymer or copolymer, a methacrylic acid derived polymer or copolymer, and a methacrylic acid ester derived polymer; and b) from about 1 to about 20 weight % of at least one water soluble organic pigment.

11. The composition of claim 10 wherein said polymeric component is present in an amount of from about 20 to about 50 percent by weight of the composition.

12. The composition of claim 11 wherein said polymeric component comprises monomeric elements having about 1 to 18 carbon atoms.

13. The composition of claim 12 wherein at least one of said monomeric elements is selected from the group consisting of methacrylate, methylmethacrylate, butylacrylate, and combinations thereof.

14. The composition of claim 13 wherein said polymeric component is ammonium acrylate.

15. A flow-through cosmetic applicator comprising the composition of claim 1.

16. The applicator of claim 15 wherein said applicator is a nib-type eyeliner pen

17. A flow-through cosmetic applicator comprising the composition of claim 10.

18. The applicator of claim 17 wherein said applicator is a nib-type eyeliner pen.

19. A method of preparing a long-wearing cosmetic composition comprising combining a polymeric component selected from the group consisting of an acrylic acid derived polymer or copolymer, an acrylic acid ester derived polymer or copolymer, a methacrylic acid derived polymer or copolymer, and a methacrylic acid ester derived polymer or copolymer, and a water soluble organic pigment.

20. A method according to claim 19 wherein the polymer comprises monomeric elements having about 1 to 18 carbon atoms.

21. A method according to claim 20 wherein at least one of the monomeric elements is selected from the group consisting of methacrylate, methylmethacrylate, butylacrylate, and combinations thereof.

22. A method of preparing a long-wearing cosmetic composition for use in a flow-through nib-type eyeliner pen comprising combining an ammonium acrylate copolymer and a water soluble organic pigment.
